(19) United States
(12) Patent Application Publication
(10) Pub. No.: US 2025/0160714 A1
(43) Pub. Date: May 22, 2025

(54) GARMENT SYSTEM WITH A DYNAMIC ELECTRODE LEAD CONFIGURATION

(71) Applicant: MYANT INC., Toronto (CA)

(72) Inventors: Amin MAHNAM, Toronto (CA); Bastien MOINEAU, Toronto (CA); Milad ALIZADEH-MEGHRAZI, Toronto (CA)

(21) Appl. No.: 18/839,109

(22) PCT Filed: Feb. 16, 2023
(86) PCT No.: PCT/CA2023/050199

§ 371 Date: Aug. 16, 2024

Related U.S. Application Data
(60) Provisional application No. 63/311,291, filed on Feb. 17, 2022.

(51) Int. Cl. A61B 5/256; A61B 5/00; A61B 5/26; A61B 5/27

(52) U.S. Cl. CPC A61B 5/256; A61B 5/26; A61B 5/27; A61B 5/6804; A61B 5/6825; A61B 5/74

(57) ABSTRACT

Disclosed herein are systems and methods for sensing electrophysiological signals using dynamically changing lead configurations. A garment system is provided, the system having a plurality of electrodes, each disposed to contact a respective body portion of a plurality of body portions of a wearer of the garment system. Electrophysiological signals of the wearer are sensed in a first lead configuration of the garment system using a first subset of the electrodes. At least one change in contact location is detected between the electrodes and the body portions. Upon such detecting, further electrophysiological signals of the wearer are sensed in a second lead configuration of the garment system using a second subset of the electrodes. The second lead configuration may provide an improved signal quality relative to the first lead configuration.

### CROSS-REFERENCE TO RELATED APPLICATION(S)

This application claims the benefit of and priority to U.S. provisional patent application No. 63/311,291 filed 17 Feb. 2022, the entire content of which is hereby incorporated by reference.

### FIELD

The present disclosure relates to garments, and more specifically relates to garment systems for sensing electrophysiological signals.

### BACKGROUND

Obtaining electrophysiological signals generated by an individual's body can provide useful knowledge about that individual's state. Athletes and medical patients, among a number of other users, can benefit from sensing and recording of sensor signals including electrophysiological signals from their bodies to improve their health or physical fitness management. Unfortunately, existing arrangements for detecting such signals can be inconvenient or uncomfortable. For example, such arrangements may require an individual to visit a facility of a clinical professional. For example, such arrangements may require individuals to wear measuring devices that limit motion and/or can only be used for a short duration of time.

### SUMMARY

In an aspect, there is provided a method of sensing electrophysiological signals using dynamically changing lead configurations. The method includes: providing a garment system having a plurality of electrodes, each disposed to contact a respective body portion of a plurality of body portions of a wearer of the garment system; sensing electrophysiological signals of the wearer in a first lead configuration of the garment system using a first subset of the electrodes; detecting at least one change in contact location between the electrodes and the body portions; and upon said detecting, sensing further electrophysiological signals of the wearer in a second lead configuration of the garment system using a second subset of the electrodes, where the second lead configuration provides an improved signal quality relative to the first lead configuration.

In some embodiments, the first subset of the electrodes and the second subset of the electrodes are the same.

In some embodiments, the first subset of the electrodes and the second subset of the electrodes are different.

In some embodiments, the change in contact location includes a change in contact of a given one of the electrodes from a given one of the body portions to a different one of the body portions.

In some embodiments, the change in contact location includes a hand of the body portions coming into contact with a particular one of the electrodes.

In some embodiments, said sensing electrophysiological signals includes sensing at least one of ECG, EEG, EMG, EOG, or bioimpedance signals.

In some embodiments, the method further includes establishing baseline readings for the wearer based on said sensing in the first lead configuration.

In some embodiments, the method further includes detecting a deviation from the baseline readings based on said sensing in the first lead configuration.

In some embodiments, the method further includes upon said detecting, providing a notification to the wearer to cause the garment system to transition to the second lead configuration.

In another aspect, there is provided a garment system for sensing electrophysiological signals using dynamically changing lead configurations. The system includes: a plurality of electrodes, each disposed to contact a respective body portion of a plurality of body portions of a wearer of the garment system. The garment system is operable to: sense electrophysiological signals of the wearer in a first lead configuration using a first subset of the electrodes; detect at least one change in contact location between the electrodes and the body portions; and sense further electrophysiological signals of the wearer in a second lead configuration using a second subset of the electrodes, where the second lead configuration provides an improved signal quality relative to the first lead configuration.

In some embodiments, the first subset of the electrodes and the second subset of the electrodes are the same.

In some embodiments, the first subset of the electrodes and the second subset of the electrodes are different.

In some embodiments, at least one of the electrodes is disposed in a garment pocket.

In some embodiments, at least one of the electrodes is disposed on an interior garment surface.

In some embodiments, at least one of the electrodes is disposed on an exterior garment surface.

In some embodiments, at least one of the electrodes is disposed on a flap of a garment, wherein the flap is configured to be manipulated between a first position at an interior of the garment, and a second position at an exterior of the garment.

In some embodiments, the system includes a contact surface for a hand of the wearer that is removably attachable to a garment.

In some embodiments, the system includes a controller operable to process the electrophysiological signals and the further electrophysiological signals.

In some embodiments, the controller is communicatively coupled to at least one of the electrodes by way of an electrically conductive pathway.

In some embodiments, the electrically conductive pathway is formed of conductive yarn.

In some embodiments, the plurality of electrodes includes a textile electrode formed of conductive yarn.

In various further aspects, the disclosure provides corresponding systems and devices, and logic structures such as machine-executable coded instruction sets for implementing such systems, devices, and methods.

In this respect, before explaining at least one embodiment in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

### BRIEF DESCRIPTION OF THE DRAWINGS

In the drawings, embodiments are illustrated by way of example. It is to be expressly understood that the description and drawings are only for the purpose of illustration and as an aid to understanding, and are not intended as a definition of the limits of the invention.

Embodiments will now be described, by way of example only, with reference to the attached figures, wherein:

FIG. 1 is a perspective view of a garment system, in accordance with an embodiment;

FIG. 2A and FIG. 2B are a side-elevation view and a top cross-sectional view, respectively, of the garment system of FIG. 1 worn and operated in a first lead configuration, in accordance with an embodiment;

FIG. 3A and FIG. 3B are a side-elevation view and a top cross-sectional view, respectively, of the garment system of FIG. 1 worn and operated in a second lead configuration, in accordance with an embodiment;

FIG. 4 is a schematic diagram of an ECG lead configuration for the garment system of FIG. 1 when in second lead configuration, in accordance with an embodiment;

FIG. 5 is a flowchart of an example method performed using the garment system of FIG. 1, in accordance with an embodiment;

FIG. 6A is a perspective view of another garment system, in accordance with an embodiment;

FIG. 6B is a perspective view of the garment system of FIG. 6A with clips attached to a garment, in accordance with an embodiment;

FIG. 6C is a side elevation cross-sectional view of a region of the garment system of FIG. 6A worn and operated in a first lead configuration, in accordance with an embodiment;

FIG. 6D is a side elevation cross-sectional view of a region of the garment system of FIG. 6A, in accordance with an embodiment;

FIG. 7A is a side elevation cross-sectional view of a region of the garment system of FIG. 6A, worn and operated in a second lead configuration, in accordance with an embodiment

FIG. 7B is a perspective view of the garment system of FIG. 6A, worn and operated in a second lead configuration, in accordance with an embodiment;

FIG. 8A is a perspective view of a garment system with flaps at an interior of a garment, in accordance with an embodiment;

FIG. 8B is a perspective view of the garment system of FIG. 8A with flaps at an exterior of a garment, in accordance with an embodiment;

FIG. 8C is a side elevation cross-sectional view of a region of the garment system of FIG. 8A, worn and operated in a first lead configuration, in accordance with an embodiment;

FIG. 8D is a side elevation cross-sectional view of a region of the garment system of FIG. 8A, with flaps at an exterior of a garment, in accordance with an embodiment;

FIG. 9A is a side elevation cross-sectional view of a region of the garment system of FIG. 8A, worn and operated in a second lead configuration, in accordance with an embodiment;

FIG. 9B is a perspective view of the garment system of FIG. 8A, worn and operated in a second lead configuration, in accordance with an embodiment;

FIG. 10A and FIG. 10B are each a side elevation cross-sectional view of a region of a garment system, in accordance with an embodiment;

FIGS. 11A and 11B show graphs of ECG signals, in accordance with one or more embodiments; and

FIG. 12A and FIG. 12B are a perspective view and a top cross-sectional view, respectively, of a garment system, in accordance with an embodiment.

### DETAILED DESCRIPTION

Disclosed herein are embodiments of a garment system for sensing electrophysiological signals via a plurality of electrodes, each of the electrodes disposed to contact a respective body portion of a plurality of body portions of a wearer of the garment system.

In various embodiments, the garment system is configured to change dynamically an electrode lead configuration defining a signal channel (or channels) through which the electrophysiological signals are obtained via the electrodes. In some embodiments, the garment system may effect a lead configuration change in response to a manipulation of the garment system, e.g., to cause an electrode to contact a different body portion of a wearer, or to cause an electrode that was previously not in contact with the wearer to come into contact with the wearer.

In some embodiments, the garment system may effect a lead configuration change in response to a manipulation of a body position of the wearer, e.g., to contact an electrode with a different body portion of the wearer, or to contact an electrode that was previously not in contact with any body portion of the wearer.

Conveniently, dynamic lead configuration changes provided by some embodiments of the garment systems use, opportunistically as available, a more optimal lead configuration for signal sensing, while maintaining signal sensing using a less optimal lead configuration otherwise.

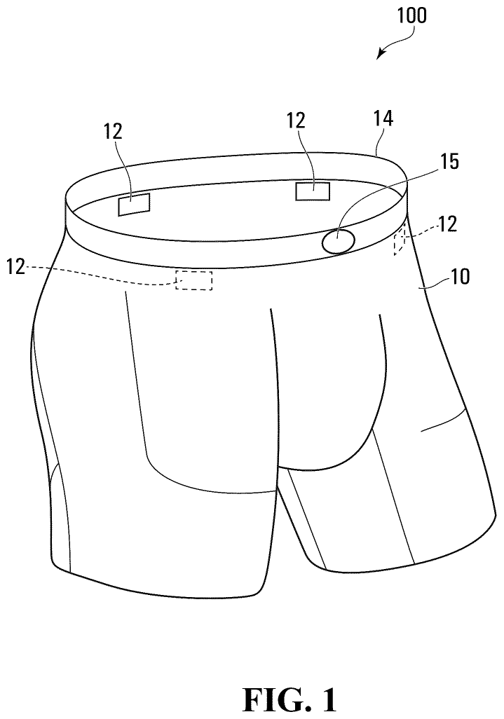

FIG. 1 depicts a garment system 100, in accordance with an embodiment. As depicted, garment system 100 includes a garment 10 in the form of underpants. Garment 10 includes a plurality of electrodes 12, each for contacting a wearer of garment 10, through which electrophysiological signals may be sensed.

The depicted embodiment is configured for sensing electrophysiological signals that are electrocardiogram (ECG) signals. However, other types of electrophysiological signals may also be sensed in other embodiments.

As shown, electrodes 12 are disposed proximate a waistband 14 of garment 10, with two electrodes 12 disposed at a front side of garment 10 and two electrodes 12 disposed at a rear side of garment 10. Of these electrodes 12, two are disposed at a right side of garment 10, while two are disposed at a left side of garment 10.

Electrodes 12 are each disposed on an interior surface of garment 10 to contact a wearer of garment 10. Each electrode 12 may be disposed on garment 10 such that, when it is worn, electrode 12 contacts a specific body portion of the wearer. For example, when the garment 10 is underpants, electrodes 12 may be positioned on garment 10 such that they contact, respectively, a left front pelvis region, a right front pelvis region, a left rear pelvis region, and a right rear pelvis region. Of course, electrodes 12 may also be positioned on garment 10 to contact any other particular body portion of wearer 200 suitable for obtaining desired electrophysiological signals.

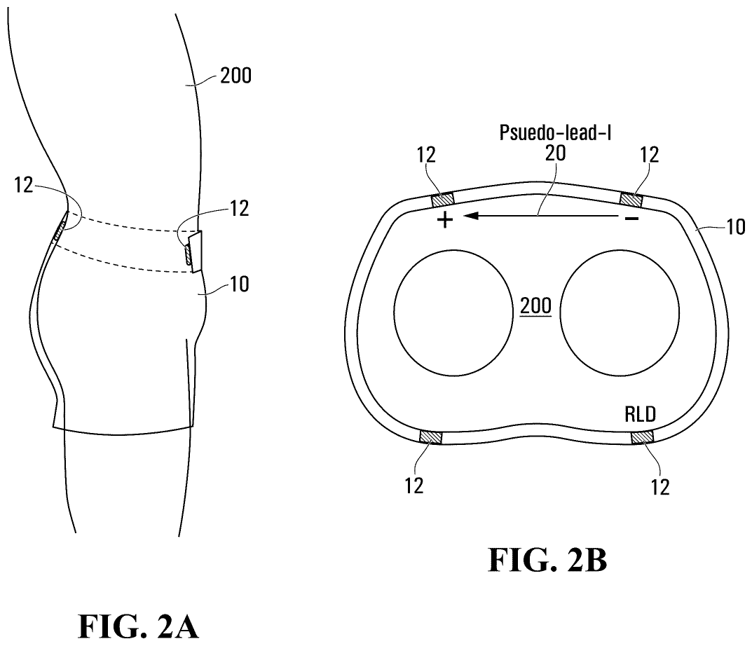

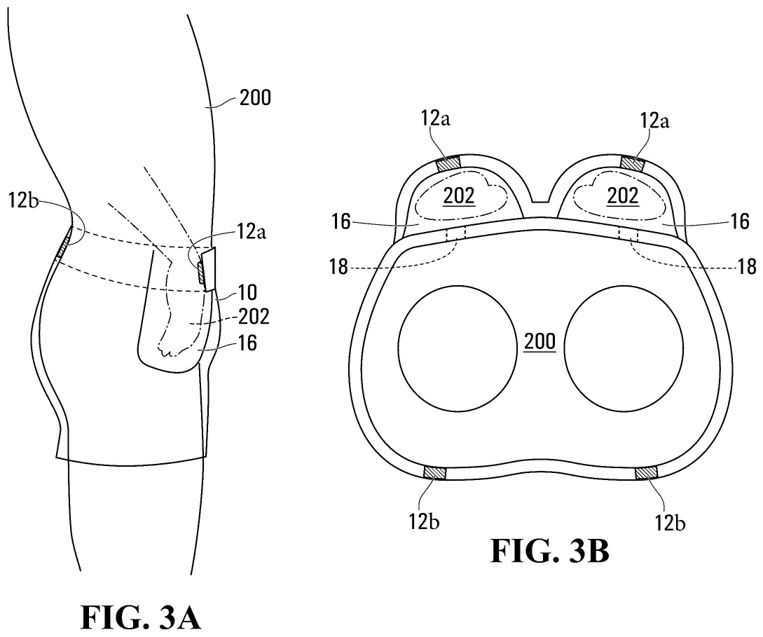

In the depicted embodiment, the particular lead configuration used to obtain ECG signals via garment system 100 depends on how garment 10 is worn. FIG. 2A and FIG. 2B are a side-elevation view and a top cross-sectional view, respectively, garment 10 worn by a wearer 200 to provide a first lead configuration. FIG. 3A and FIG. 3B are a side-elevation view and a top cross-sectional view, respectively, garment 10 worn by wearer 200 to provide a second lead configuration.

Referring now to the first lead configuration shown in FIG. 2A and FIG. 2B, each electrode 12 contacts a body portion of wearer 200 proximate the wearer's pelvis. In this first lead configuration, two electrodes 12 at the front of wearer 200 function as positive and negative electrodes establishing a pseudo-lead for ECG sensing. Such a lead obtains signals corresponding to a Lead I from a conventional 3-lead configuration for sensing ECG signals. In various embodiments, other pseudo-leads may be established using a channel established through other combinations of electrodes 12. Such other pseudo-leads may, for example, correspond to a Lead II or Lead III from a conventional 3-lead ECG sensing configuration. In some embodiments, an electrode 12 functions as a ground electrode.

Optionally, an electrode 12 at a rear side of wearer 200 functions as a right-leg-drive (RLD) electrode. Providing such an RLD electrode may reduce common-mode interference.

Reference is now made to the second lead configuration shown in FIG. 3A and FIG. 3B. As shown, wearer 200 has inserted hand(s) 202 into pockets 16 of garment 10. This results in a change in contact locations between electrodes 12a in the front of garment 10 and wearer 200. In particular, when hand(s) 202 are inserted into pockets 16, those electrodes 12a which previously contacted respective front pelvis regions wearer 200 now contact a respective hand 202. Electrodes 12b at the back of garment 10 each contacts a body portion of wearer 200 at the rear side of wearer 200. Both electrodes 12a at the front of garment 10 and electrodes 12b at the back of garment 10 may be referred to as electrodes 12.

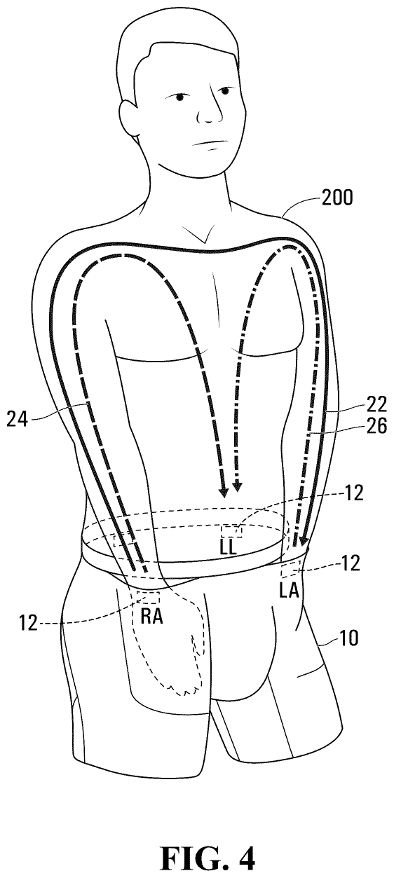

This second lead configuration provides a lead configuration approximating a conventional 3-lead configuration for sensing ECG signals. As shown in FIG. 4, electrodes 12 contacting hand(s) 202 provide, respectively, a right arm (RA) electrode and a left arm (LA) electrode. Meanwhile, electrode 12 contacting a left rear pelvis region of wearer 200 provides a left leg (LL) electrode. These electrodes provide lead 22 (ECG Lead I), lead 24 (ECG Lead II), and lead 26 (ECG Lead III).

As best seen FIG. 3B, each pocket 16 includes an electrode 12a on an interior surface at a front side of pocket 16. Each pocket 16 includes an aperture 18 on a rear interior surface of pocket 16 that connects a pocket cavity (for receiving a hand 202, among other things) with a pelvis region of wearer 200. When hand 202 is not inserted in pocket 16, electrode 12a contacts the pelvis region of wearer 200 by way of aperture 18 (e.g., as shown in FIG. 2A and FIG. 2B). Conveniently, by providing aperture 18 as shown, the same electrodes 12a at the front of garment 10 are used in both the first lead configuration and in the second lead configuration.

Wearer 200 can cause garment system 100 to transition from the first lead configuration to the second lead configuration by inserting hands 202 into respective pockets 16 to contact electrodes 12 disposed therein.

When garment system 100 operates in the first lead configuration, it can obtain electrophysiological signals for a substantial portion of a given time period (e.g., an hour, a day, etc.), which may be substantially an entire duration that garment system 100 is worn. In such circumstance, garment system 100 may be suitable for use to sense electrophysiological signals on a continuous or pseudo-continuous basis. When electrophysiological signals are sensed on a continuous or pseudo-continuous basis, data may be obtained for determining physiological statistics of the wearer, such as through sensing a plurality of readings over time (e.g., periodically). In some scenarios, baseline readings for wearer 200 may be obtained upon processing sensed electrophysiological signals. In some scenarios, trends and deviations from the baseline readings may be determined from sensed electrophysiological. In one example, deviations may include arrhythmia detected upon processing ECG signals. In one example, deviations may include atrial fibrillation detected by monitoring a consistency of a P-wave that represents atrial activity. In other examples, deviations may be detected upon monitoring a QT duration (e.g., a time from the start of the Q wave to the end of the T wave). In other examples, deviations may be detected upon monitoring ST elevation. Conveniently, embodiments of garment system 100 allow such deviations to be monitored throughout the course of a day, e.g., for as long as garment system 100 is worn.

When garment system 100 operates in the second lead configuration, electrophysiological signals are sensed in a manner approximating a conventional 3-lead configuration for sensing ECG signals. Consequently, in some embodiments, signal quality in the second lead configuration may be improved (e.g., having a higher signal-to-noise ratio (SNR)) compared to the signal quality in the first lead configuration using one or more pseudo-leads.

Wearer 200 can cause garment system 100 to transition from operating in the first lead configuration to operating in the second lead configuration, and vice versa. For example, wearer 200 may desire to transition garment system 100 from the first lead configuration to the second lead configuration when prompted by garment system 100, e.g., when a deviation in a state of wearer 200 has been detected requiring confirmation using signals of improved quality. Such a deviation may, for example, be an arrhythmia detected based on processing signals obtained when operating in the first lead configuration as further detailed below. Accordingly, garment system 100 may provide a suitable notification to wearer 200 upon detecting such a deviation. Garment system 100 may transmit such notifications to a computing device operated by wearer 200 (e.g., a smartphone or the like). Of course, a wearer 200 may desire garment system 100 to transition from operating in the first lead configuration to operating in the second lead configuration at any other time, e.g., when improved signal quality associated with a three-channel measurement is desired.

In some embodiments, electrodes 12 are not placed in a pocket 16, but rather in other types of openings or folds of garment 10. In one example, electrodes 12 are placed in side pockets of a vest. In another example, electrodes 12 are placed in a front pocket of a hooded sweater. Electrodes 12 may be disposed in a garment 10 to facilitate various manners of contact with hands 202 for operating in the second lead configuration. Electrodes 12 may be disposed to face towards the body of wearer 202 or away from the body of wearer 200.

Referring again to FIG. 1, in the depicted embodiment, garment system 100 also includes a controller 15 disposed on garment 10. Controller 15 can include various electronics components such as, for example, a memory for storing data and processor-executable instructions and data reflective of recorded signals sensed via electrodes 12, a processor for executing stored instructions, and a communication interface for sending or receiving data.

The processor of controller 15 may be a microprocessor or microcontroller, a digital signal processing (DSP) processor, an integrated circuit, a field programmable gate array (FPGA), a reconfigurable processor, a programmable read-only memory (PROM), or combinations thereof. The memory of controller 15 may include a computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM). The communication interface of controller 15 is an interface for communication by way of a wireless (e.g., Bluetooth or WiFi) or wired link.

Controller 15 is communicatively coupled (e.g., by way of its communication interface) to each electrode 12, to receive sensor signals therefrom. In some embodiments, controller 15 may be communicatively coupled to one or more electrodes 12 by way of electrically conductive pathways formed of conductive fibres of garment 10. In some embodiments, controller 15 may be communicatively coupled to one or more electrodes 12 by way of a wireless link. Sensor signals may be received at controller 15 in analog or digital form.

In some embodiments, controller 15 processes electrophysiological signals received from one or more signal channels. In one example, controller 15 may process ECG signals to estimate a heart rate or other heart characteristics of wearer 200.

In some embodiments, controller 15 transmits electrophysiological signals received from one or more signal channels to a remote destination at, for example, another computing device (e.g. a smartphone operated by wearer 200 on other similar device) or to a cloud repository. Such signals may be transmitted in raw format and/or in preprocessed format, with such preprocessing performed at controller 15. Such signals may be transmitted to such other computing device for viewing and/or further processing of the electrophysiological signals. In one example, the other computing device may display signals via a graphical user interface for viewing by wearer 200 or by another person other than the wearer that has been provided access to the signals.

In the depicted embodiment, controller 15 is mounted in a suitable housing, for example, on an exterior surface (i.e., outward facing relative to wearer 200) of a garment 10. In some embodiments, controller 15 may be mounted on an interior surface of a garment 10. In some embodiments, controller 15 may be disposed in a pocket or other cavity or enclosure of the garment 10. Controller 15 may also include a power supply (e.g., a battery) for supplying power to one or more components of controller 15, as well to electrodes 12 as required.

In some embodiments, at least a portion of controller 15 is removably attached to a garment 10. For example, at least a portion of controller 15 may be removed, for example to recharge its components (e.g., its power supply), or to be augmented or otherwise changed (e.g., to update firmware), and so forth. In some embodiments, at least a portion of controller 15 may be removed, for example, when the garment 10 is cleaned or laundered.

In some embodiments, garment 10 may be a different type of garment for covering one or more different body portions, such as a leg or arm, torso/trunk, buttocks, foot or ankle, wrist or hand, a neck, a head, or the like. So, for example, the garment 10 may be a shirt, a pair of pants, a waistband, a chest band, or the like.

In some embodiments, garment system 100 may include multiple garments 10, each for wearing on one or more body portions, such as, for example, a waist, an abdomen, a limb, or the like. The multiple garments 10 may operate in concert. For example, electrodes 12 may be disposed in multiple garments 10, yet operate as part of a single system, e.g., with all electrodes 12 connected for data communication (e.g., via electrically conductive pathways or via wireless communication) with a single controller or a single computing device. In one specific example, some electrodes 12 are positioned on a waistband, and others on a chest band.

In some embodiments, garment 10 includes one or more resilient or elastic elements to provide a mechanical biasing force that causes contact between an electrode 12 and wearer 200 to be maintained. In some embodiments, garment 10 is shaped to provide maintained contact between an electrode 12 and wearer 200.

### Operation

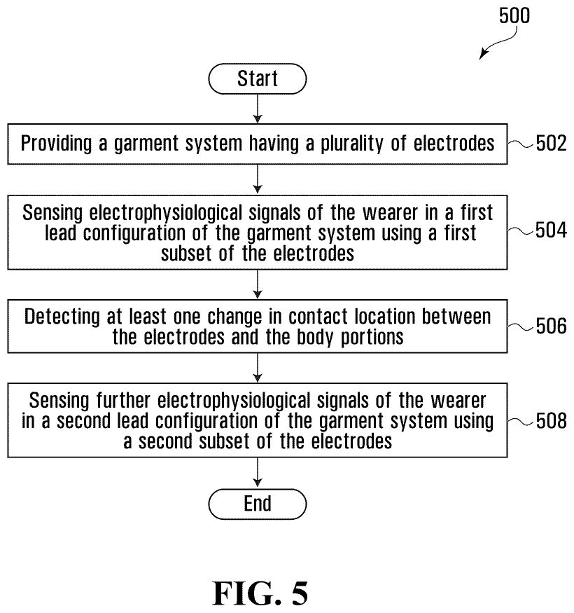

The operation of garment system 100 is further described with reference to an example method depicted in FIG. 5, in accordance with an embodiment. During operation, garment system 100 can be operated to perform the depicted method of sensing electrophysiological signals using dynamically changing lead configurations.

In accordance with the depicted method, a garment system 100 is provided at block 502. Garment system 100 has a plurality of electrodes 12, each disposed to contact a respective body portion of a plurality of body portions of a wearer 200 of garment system 100.

At block 504, electrophysiological signals of wearer 200 are sensed in a first lead configuration of garment system 100 using a first subset of electrodes 12.

At block 506, at least one change in contact location between electrodes 12 and the body portions is detected. In some embodiments, a change in contact location and a body portion is detected by detecting a change in characteristics of a particular electrode 12 or change in signals obtained from a particular electrode 12, e.g., a change in impedance, a change in signal amplitude, a change in signal-to-noise ratio (SNR), or the like. In some embodiments, a change in contact location and a body portion is detected by detecting a particular change in posture of wearer 200 using, for example, a suitable motion sensor and/or strain gauge sensor incorporated into a garment 10.

At block 508, upon detecting the change(s) in contact location, electrophysiological signals of wearer 200 are sensed in a second lead configuration of the garment system using a second subset of electrodes 12. In some embodiments, the second lead configuration provides an improved signal quality relative to the first lead configuration. Improved signal quality may be characterized, for example, as improved SNR, higher signal amplitude, or other desirable signal attribute.

As used herein, the term “subset” refers to one or more of electrodes 12, up to and including all electrodes 12 in garment system 100. So, each of the first subset of electrodes and the second subset of electrodes includes at least one electrode, and may include up to all electrodes 12 (e.g., a full set).

In some embodiments, depending on the configuration of garment system 100, the arrangement of electrodes 12, and the type of change in contact that triggers a change from the first lead configuration to the second lead configuration, the first subset of the electrodes and the second subset of the electrodes differ. In other embodiments, the first subset of the electrodes and the second subset of the electrodes are the same.

It should be understood that steps of one or more of the blocks depicted in FIG. 5 may be performed in a different sequence or in an interleaved or iterative manner. Further, variations of the steps, omission or substitution of various steps, or additional steps may be considered.

### Additional Embodiments—Clips and Flaps

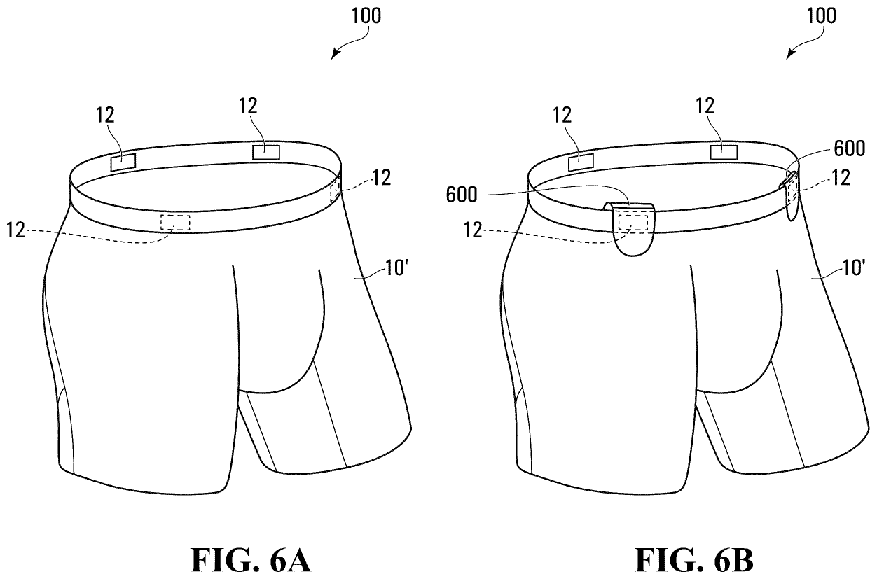

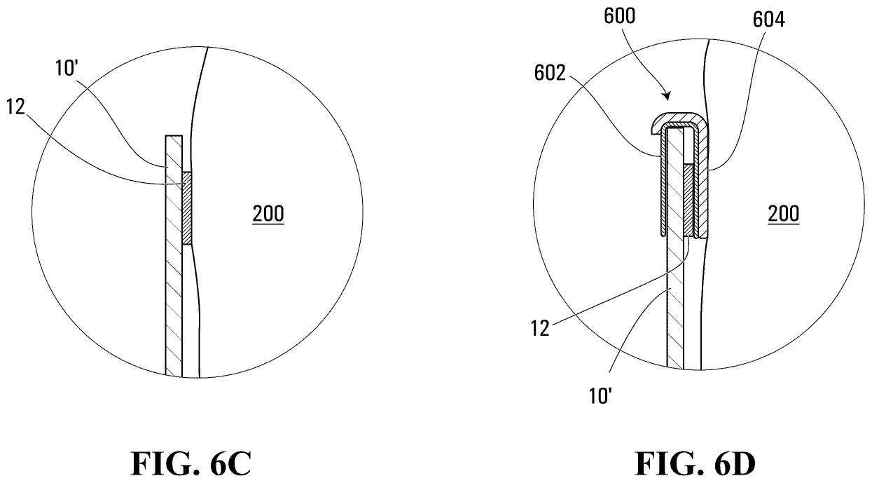

FIG. 6A and FIG. 6B each is a perspective view of a garment system 100, in accordance with another embodiment. As depicted, garment system 100 includes a garment 10′ and two clips 600. Garment 10′ includes four electrodes 12 disposed on an interior surface of garment 10′ to contact, respectively, a left front pelvis region, a right front pelvis region, a left rear pelvis region, and a right rear pelvis region of wearer 200. Clip 600 can be manipulated by wearer 200 to be removably attached to garment 10′ at a location of an electrode 12, e.g., to be clipped to the top of a waistband or other portion of garment 10′. When attached to the garment 10′, clip 600 is coupled to the particular electrode 12 at the clipped location, in manners detailed below. Clip 600 may include isolating fabric, while garment 10′ may include conductive fabric (e.g., conductive fibres).

FIG. 6C is a side elevation cross-sectional view of a region of garment system 100 when clips 600 are not attached to garment 10′. As shown, electrodes 12 at the front of garment 10′ contacts front pelvis regions, respectively, of wearer 200.

FIG. 6D is a side elevation cross-sectional view of a region of garment system 100 when at least one clip 600 is attached to garment 10′. As depicted, electrodes 12 at the front of garment 10′ do not contact front pelvis regions of wearer 200. In particular, these electrodes 12 are insulated from pelvis regions of wearer 200 by clip 600, by interposing an electrical insulator 604 of clip 600 between the electrodes 12 and the pelvis regions. In some embodiments, the electrical insulator 604 may include isolating fabric.

As depicted, clip 600 includes a substantially U-shaped electrical conductor 602 that extends from an interior of garment 10′ to an exterior of garment 10′. The portion of electrical conductor 602 at the interior of garment 10′ contacts electrode 12 to establish electrical communication therewith. Meanwhile, the portion of electrical conductor 602 at the exterior of garment 10′ provides a contact location for a hand 202 of wearer 200. Clip 600 also includes an electrical insulator 604 that extends at least along the portion of clip 600 on the interior of garment 10′. As depicted, electrical insulator 604 also extends along the top of clip 600 that rests on a waistband region of garment 10′. Electrical insulator 604 is located and sized to cover a corresponding electrode 12 and thereby provide electrical insulation between that electrode 12 and a corresponding pelvis region of wearer 200.

Electrical conductor 602 is formed of an electrically conductive material. Such conductive material may include, for example, conductive fabric formed of conductive yarn. Electrical insulator 604 is formed of an electrically insulating material. Such electrically insulating material may, for example, include a plastic, a rubber, or the like.

In some embodiments, a clip 600 may include a suitable combination of flexible materials (such as a fabric) and semi-rigid materials (such as a plastic). Such combination of materials may be selected to provide a degree of flex that allows clip 600 to be pressed around a portion of garment 10′, e.g., to maintain contact (and electrical communication) electrical conductor 602 and the electrode 12.

In some embodiments, clips 600 are mechanically connected to one another and spaced by such mechanical connection to align clips 600 with respective electrodes 12.

Garment system 100 is operable in a first lead configuration when clips 600 are removed from garment 10′ (FIG. 6A and FIG. 6C), and is operable in a second lead configuration when clips 600 are attached to garment 10′ (FIG. 6B and FIG. 6D).

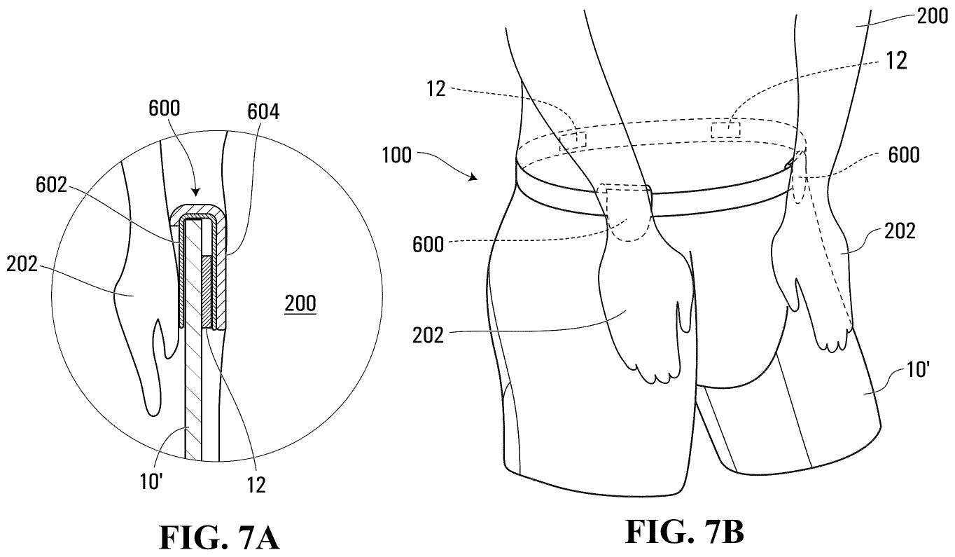

Garment 10′ differs from garment 10 in how a wearer 200 can cause garment system 100 to transition from a first lead configuration to a second lead configuration. As noted above, a wearer 200 of garment 10 can cause a transition of garment system 100 from a first lead configuration to a second lead configuration by inserting hands 202 into pockets 16. In contrast, a wearer 200 of garment 10′ can cause a transition of garment system 100 from a first lead configuration to a second lead configuration by attaching clips 600 and then placing hands 202 on respective clips 600, as depicted in FIG. 7A and FIG. 7B. This represents a change in contact location between the electrodes and the body portions of wearer 200, namely, each electrode 12 coupled to a clip 600 changes from contacting a pelvis region to contacting a hand 202. Conversely, wearer 200 can cause a transition of garment system 100 from the second lead configuration to the first lead configuration by removing hands 202 from respective clips 600, and then detaching clips 600 from garment 10′. This represents a reverse change in contact location between electrodes 12 and the body portions of wearer 200.

As best seen in FIG. 7A and FIG. 7B, hands 202 are in contact with respective electrical conductors 502 when garment system 100 is in the second lead configuration. This provides a lead configuration approximating a conventional 3-lead configuration for sensing ECG signals. As depicted in FIG. 4. Otherwise, garment system 100 provides a pseudo-lead for ECG sensing, substantially similar to that described in connection with FIG. 2A and FIG. 2B.

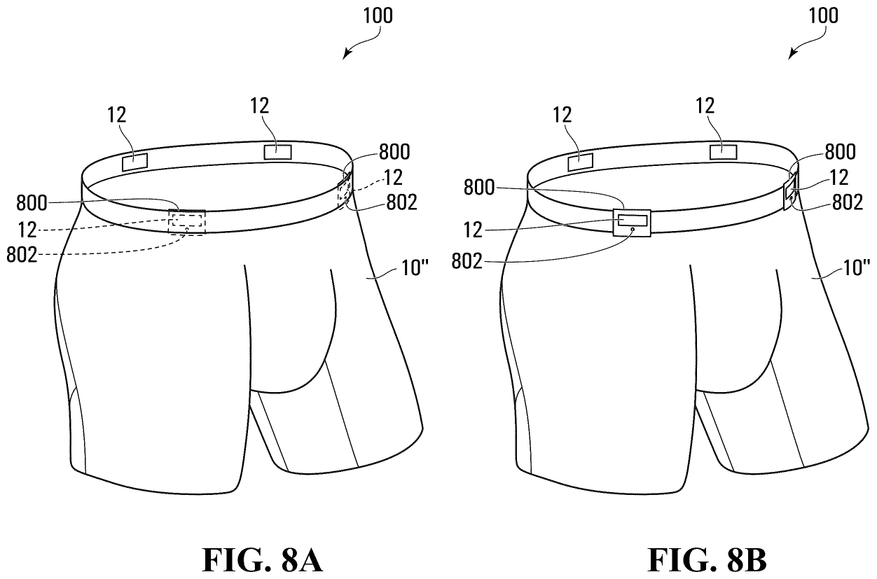

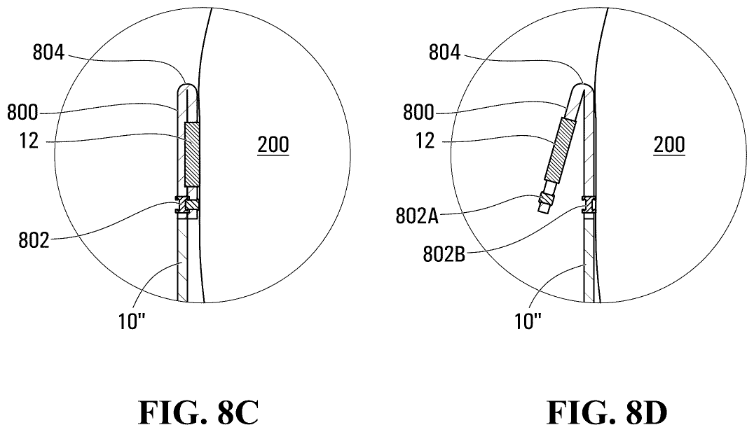

FIG. 8A and FIG. 8B each is a perspective view of a garment system 100, in accordance with another embodiment. As depicted, garment system 100 includes a garment 10″. Garment 10″ includes two flaps 800, which can each be manipulated (e.g., by wearer 200) to alternate between a first position at an interior of garment 10″ (FIG. 8A) and a second position at an exterior of garment 10″ (FIG. 8B). Garment 10″ includes two electrodes 12 disposed proximate a waistband at the rear of garment 10″. Garment 10″ also includes two electrodes 12, each disposed, respectively, on a respective flap 800. Each flap 800 includes a hinge 804, about which flap 800 can be flipped between the noted first and second positions.

In some embodiments, electrode 12 disposed on flap 800 is formed as a double-sided electrode. This provides an electrode surface for contacting wearer 200 on opposing faces of flap 800, and permits contact between electrode 12 and a corresponding body portion of wearer 200 regardless of whether flap 800 is in its first or second position. In other words, electrode 12 may be inverted when flap 800 is flipped about hinge 804 and yet continue to present an electrode surface to contact a corresponding body portion of wearer 200.

In some embodiments, flap 800 includes electrically conductive pathways interconnecting an electrode 12 disposed on flap 800 with other portions of the garment 10″, such as, for example, with controller 15.

In some embodiments, flap 800 is formed of a textile material. In some embodiments, flap 800 may be formed (e.g., knitted, woven, or the like) to be integral with the garment 10″ (e.g., as part of a waistband). In some embodiments, flap 800 may be formed of a semi-rigid material.

FIG. 8C is a side elevation cross-sectional view of a region of garment system 100 when flaps 800 are flipped to an interior of garment 10″. As depicted, when flaps 800 are in this position, electrodes 12 disposed on flaps 800 contact right and left front pelvis regions, respectively, of wearer 200.

FIG. 8D is a side elevation cross-sectional view of a region of garment system 100 when flaps 800 are flipped to an exterior of garment 10″. As depicted, when flaps 800 are in this position, electrodes 12 disposed on flaps 800 do not contact front pelvis regions of wearer 200.

Garment 10″ also includes snaps 802, each of which allows a flap 800 to be fastened in place when flipped to an interior of garment 10″. As depicted in FIG. 8C and FIG. 8D, flap 800 may be fastened to an interior surface of garment 10″ by mechanically connecting corresponding male portion 802A and female portion 802B of snap 802.

Garment system 100 is operable in a first lead configuration when flaps 800 are in a first position at an interior of garment 10″ (FIG. 8A and FIG. 8C), and is operable in a second lead configuration when flaps 800 are in a second position at an exterior of garment 10″ (FIG. 8B and FIG. 8D).

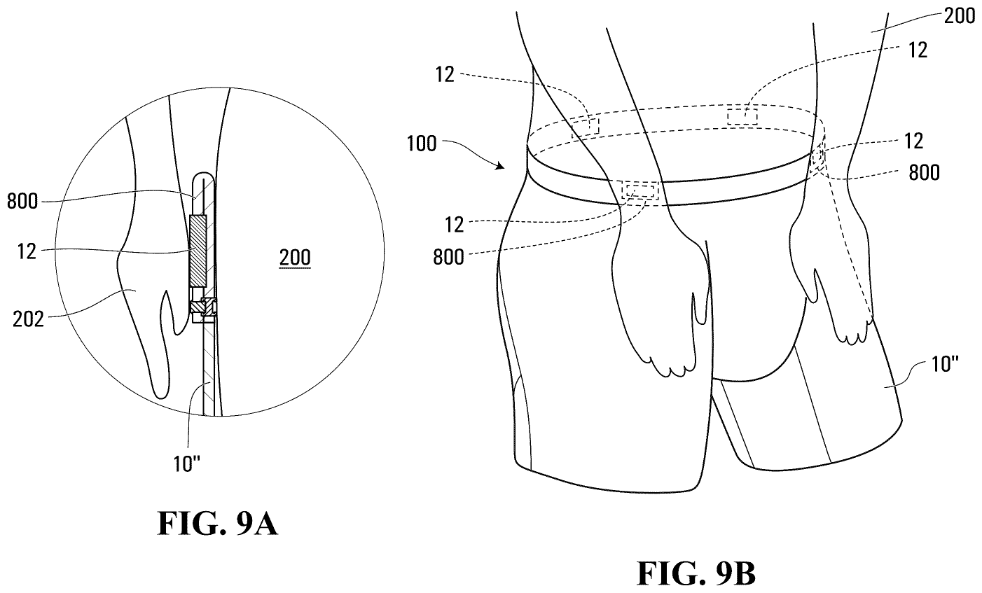

Garment 10″ differs from garment 10 in how a wearer 200 can cause garment system 100 to transition from a first lead configuration to a second lead configuration. As noted above, a wearer 200 of garment 10 can cause a transition of garment system 100 from a first lead configuration to a second lead configuration by inserting hands 202 into pockets 16. In contrast, a wearer 200 of garment 10″ can cause a transition of garment system 100 from a first lead configuration to a second lead configuration by manipulating flaps 800 to be in the second position at the exterior of garment 10″, and then placing hands 202 on respective electrodes 12 now exposed at the exterior of garment 10″. As depicted in FIG. 9A and FIG. 9B, this represents a change in contact location between the electrodes 12 and the body portions of wearer 200, namely, each electrode 12 disposed on flaps 800 from contacting a pelvis region is changed to contacting a hand 202. Conversely, wearer 200 can cause a transition of garment system 100 from the second lead configuration to the first lead configuration by removing hands 202 from electrodes 12 on flaps 800, and then manipulating flaps 800 to be in the first position at the interior of garment 10′. This represents a reverse change in contact location between electrodes 12 and the body portions of wearer 200.

As best seen in FIG. 9A and FIG. 9B, hands 202 are in contact with respective electrodes 12 disposed on flaps 800 when garment system 100 is in the second lead configuration. This provides a lead configuration approximating a conventional 3-lead configuration for sensing ECG signals, as depicted in FIG. 4. Otherwise, garment system 100 provides a pseudo-lead for ECG sensing, substantially similar to that described in connection with the embodiment shown in FIG. 2A and FIG. 2B.

Garment 10″ is otherwise substantially similar to garment 10′.

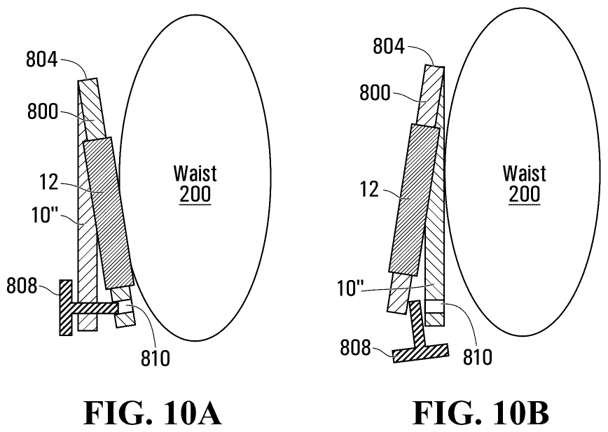

In other embodiments, flaps 800 are secured into the noted first and/or second positions via other various fastening mechanisms. For example, the fastening mechanism may include a button 808 allowing flap 800 to be secured to an interior surface of garment 10″ when flap 800 is flipped to the interior of garment 10″ (as depicted in FIG. 10A). For example, button 808 may be inserted into a corresponding slot 810 on garment 10″. Conversely, button 808 may be removed for slot 810 to unfasten flap 800 (as depicted in FIG. 10B).

Fastening mechanisms that fasten flaps 800 into a position at the interior of garment 10″ may, for example, prevent flaps 800 from coming loose during movement of wearer 200 and disrupting contact between electrode 12 and respective body portions (e.g., pelvis regions) of wearer 200.

### Example Data

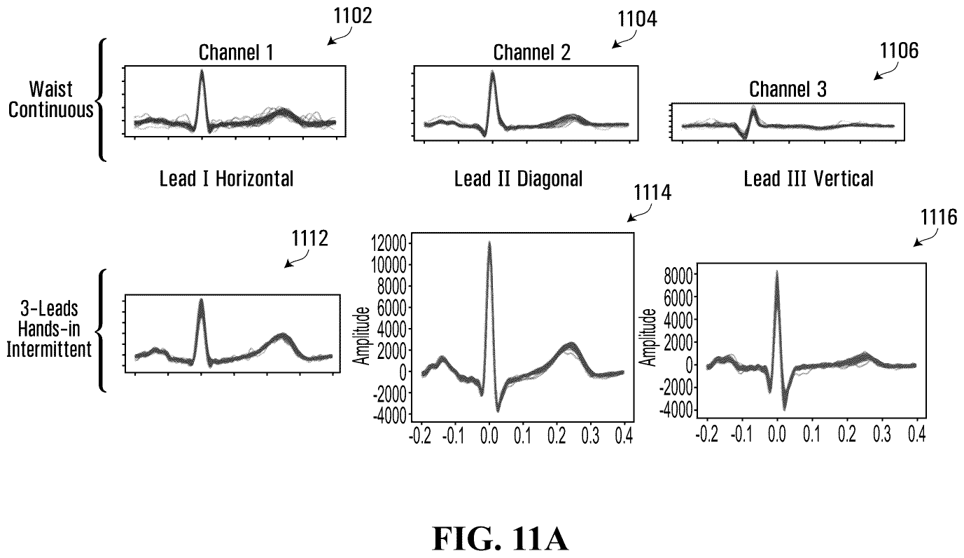

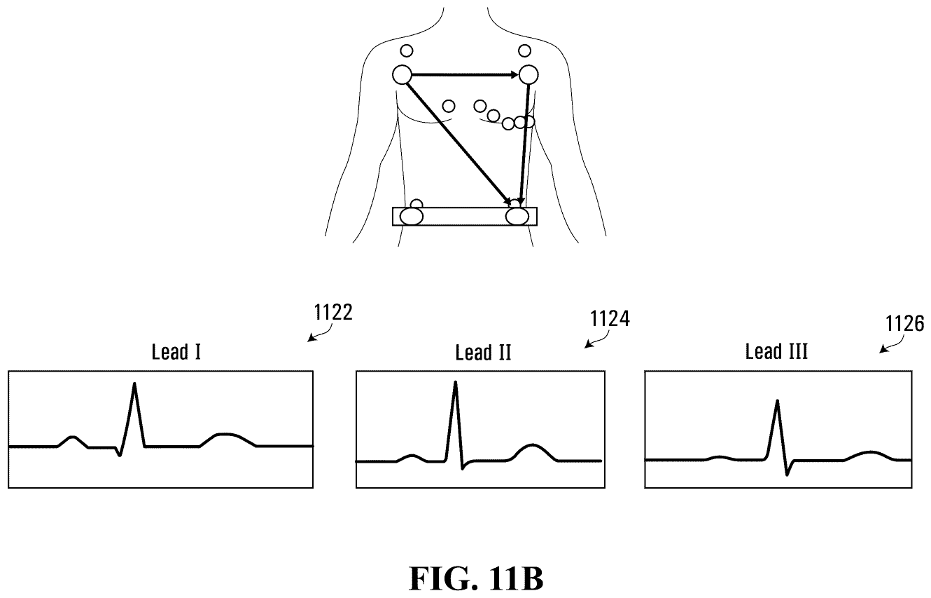

FIGS. 11A and 11B show example ECG data obtained using a garment system 100 (e.g., as depicted in FIG. 1).

Referring to FIG. 11A, graphs 1102, 1104, and 1106 show ECG signals obtained when garment system 100 operates in the first lead configuration, in which:
1) graph 1102 shows ECG signals obtained from a channel from the front left of the pelvis to the front right of the pelvis (a pseudo-lead corresponding to ECG Lead I);
2) graph 1104 shows ECG signals obtained from a channel from the front right of the pelvis to the back left of the pelvis (a pseudo-lead corresponding to ECG Lead II); and
3) graph 1106 shows ECG signals obtained from a channel from the front left of the pelvis to the back left of the pelvis (a pseudo-lead corresponding to ECG Lead III).

Still referring to FIG. 11A, graphs 1112, 1114, and 1116 show ECG signals obtained when garment system 100 operates in the second lead configuration, in which:
1) graph 1112 shows ECG signals obtained from a channel between right and left hands 202 (corresponding to ECG Lead I);
2) graph 1114 shows ECG signals obtained from a channel from right hand 202 to the back left of the pelvis (corresponding to ECG Lead II); and
3) graph 1116 shows ECG signals obtained from a channel from the left hand 202 to the back left of the pelvis (corresponding to ECG Lead III).

In FIG. 11B, Graphs 1122, 1124, and 1126 show expected ECG waveforms for Leads I, II, and III, respectively, as would be obtained using a conventional 3-lead configuration for sensing ECG signals.

### Additional Embodiments

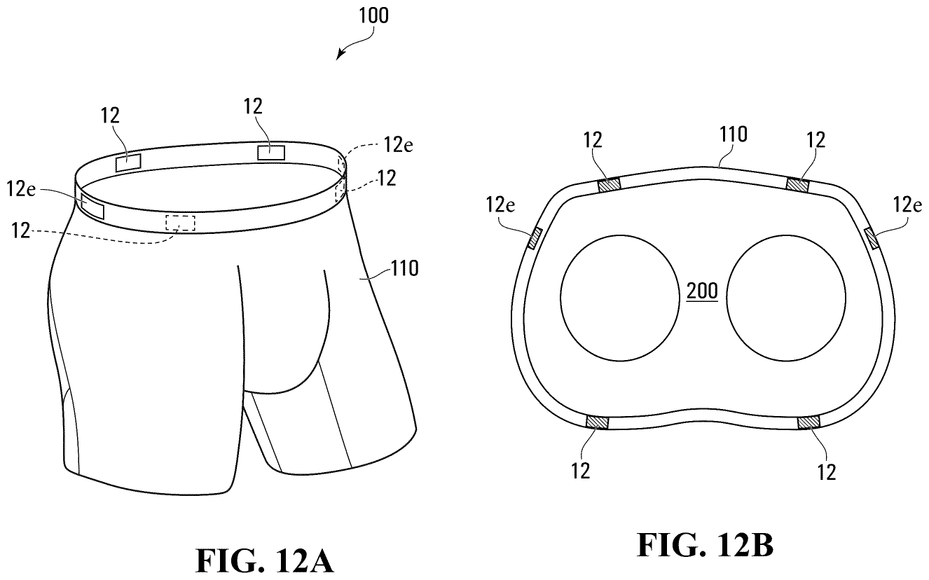

FIG. 12A and FIG. 12B are a perspective view and a top cross-sectional view, respectively, of a garment system 100, in accordance with an embodiment.

Whereas example embodiments of garment systems 100 have been described having four electrodes, garment system 100 shown in FIG. 12A and FIG. 12B includes a garment 110 with six electrodes. As depicted, garment 110 includes four electrodes disposed on an interior surface of garment 10 to contact, respectively, a left front pelvis region, a right front pelvis region, a left rear pelvis region, and a right rear pelvis region of wearer 200. Garment 110 additional includes two electrodes 12 disposed on an exterior surface of garment 110 to contact, respectively, hands 202 of wearer 200, which may be referred to as electrodes 12e for ease of reference. Electrodes 12e are disposed proximate a waistband of garment 110 at left and right sides of garment 110.

Garment system 100 is operable in a first lead configuration using electrodes 12 disposed in the interior of garment 110, substantially as described for the first lead configuration of garment 10. Garment system 100 is operable in a second lead configuration using electrodes 12 disposed at the rear interior of garment 110 in combination with electrodes 12e disposed at the front exterior of garment 110. A wearer 200 of garment 110 can cause a transition of garment system 100 from a first lead configuration to a second lead configuration by placing hands 202 on electrodes 12e. This represents a change in contact location between the electrodes and body portions of wearer 200, namely, hands 202 come into contact with electrodes 12e. Conversely, wearer 200 can cause a transition of garment system 100 from the second lead configuration to the first lead configuration by removing hands 202 from electrodes 12e.

Of note, the particular subset of electrodes used in the first lead configuration differs from the particular subset of electrodes used in the second lead configuration.

In some embodiments, electrodes 12 may be configured to sense various types of electrophysiological signals from a wearer, e.g., having different signal characteristics (e.g., voltage, bandwidth, etc.). For example, such electrophysiological signals may include one or more of electrocardiography (ECG), electroencephalography (EEG), electromyography (EMG), electrooculography (EOG), bio-impedance analysis (BIA), or electrodermal activity (EDA) signals. Various combinations of electrodes to sense various types of electrophysiological signals are contemplated. When used for a sensing function, each electrode may be referred to as a “sensor” for convenience.

In some embodiments, one or more of electrodes 12 may be a dry contact electrode. Dry contact electrodes can be categorized according to form factor into textile electrodes, flexible film electrodes, bulk electrodes, pin-shaped electrodes, microneedles, or the like. Dry contact electrodes do not contain or require a gel layer to operate. Dry electrodes may be biocompatible, easy to use, comfortable, breathable, lightweight, flexible, washable, durable, and able to maintain good signal quality during electrophysiology testing while at rest and moving. Additionally, textile-based electrodes may be worn on various body parts by attaching them to, or integrating them into, different articles of clothing such as waistbands, sleeves, pants, and headbands. In some embodiments, the dry contact electrode is a dry contact electrode as described in International Patent Publication No. WO2021/134131, the entire contents of which are hereby incorporated by reference herein.

In some embodiments, electrodes 12 are disposed on the garment 10 to avoid biological signal obstacles, such as a pelvis bone, spine, etc.

In some embodiments, electrodes 12 include electrodes adapted for use with specific body portions and/or the specific electrophysiological signals originating or traveling through those body portions.

In some embodiments, a sufficient number of electrodes 12 are disposed in a garment system 100 to establish at least one signal channel for sensing a desired type of electrophysiological signal. Such a signal channel may be formed, in one example, by electrodes serving as a positive terminal, a negative terminal, and a ground terminal, respectively. Various combinations of electrodes suitable to form a desired signal channel would be apparent to one of ordinary skill in the art.

In some embodiments, transitioning a garment system 100 between a first lead configuration and a second lead configuration includes activating at least one electrode 12. For example, in response to detecting contact between an electrode 12 and wearer 200, controller 15 may cause additional power to be supplied to that electrode 12 and/or associated electronics. For example, controller 15 may cause power to be supplied to a signal amplifier upon detecting a electrophysiological signal above a threshold from an electrode 12, the threshold being indicative of wearer 200 contacting that electrode 12.

In some embodiments, garment system 100, under control of controller 15, may selectively power an electrode 12 depending on the desired type of signal to be measured. For example, garment system 100 may switch between obtaining bioimpedance signals and obtaining ECG signals via an electrode 12, and selectively power electrode 12 accordingly.

In some embodiments, garment system 100 may include various other types of sensors for measuring a wearer state or an environmental state. For example, such other types of sensors may include, for example, a temperature sensor (to measure a skin temperature or ambient temperature), a strain gauge to measure breathing, motion sensors such as inertia measurement units, accelerometers, gyroscopes, or the like, to measure movement and posture. In some embodiments, signals sensed from electrodes 12 can be processed in combination with signals from the other types of sensors to calculate various biometric state inferences regarding the wearer such as a type or an intensity of physical activity.

In some embodiments, some or all of electrodes 12 may be used as actuators to inject electrical current/voltage to the body, e.g. for Functional Electrical Stimulation (FES) to inject electrical pulses to activate muscles.

In some embodiments, garment system 100 includes one or more garment formed of a knitted textile. In some embodiments, garment system 100 includes one or more garment formed of other textile forms and/or techniques such as weaving, knitting (warp, weft, etc.), or the like. In some embodiments, garment system 100 includes one or more garments formed of at least one of a knitted textile, a woven textile, a cut and sewn textile, a knitted fabric, a non-knitted fabric, in any combination and/or permutation thereof. Example structures and interlacing techniques of textiles formed by knitting and weaving are disclosed in U.S. patent application Ser. No. 15/267,818, the entire contents of which are herein incorporated by reference.

As used herein, “textile” refers to any material made or formed by manipulating natural or artificial fibres to interlace to create an organized network of fibres. Generally, textiles are formed using yarn, where yarn refers to a long continuous length of a plurality of fibres that have been interlocked (i.e., fitting into each other, as if twined together, or twisted together). Herein, the terms fibre and yarn are used interchangeably. Fibres or yarns can be manipulated to form a textile according to any method that provides an interlaced organized network of fibres, including but not limited to weaving, knitting, sew and cut, crocheting, knotting and felting.

Different sections of a textile can be integrally formed into a layer to utilize different structural properties of different types of fibres. For example, conductive fibres can be manipulated to form networks of conductive fibres and non-conductive fibres can be manipulated to form networks of non-conductive fibers. These networks of fibres can comprise different sections of a textile by integrating the networks of fibres into a layer of the textile. The networks of conductive fibres can form one or more conductive pathways that electrically connect electrodes or other electronic components embedded in garment 10, for conveying data and/or power to and/or from these components.

In some embodiments, multiple layers of textile may be stacked upon each other to provide a multi-layer textile.

As used herein, “interlace” refers to fibres (either artificial or natural) crossing over and/or under one another in an organized fashion, typically alternately over and under one another, in a layer. When interlaced, adjacent fibres touch each other at intersection points (e.g., points where one fibre crosses over or under another fibre). In one example, first fibres extending in a first direction can be interlaced with second fibres extending laterally or transverse to the fibres extending in the first connection. In another example, the second fibres can extend laterally at 90° from the first fibres when interlaced with the first fibres. Interlaced fibres extending in a sheet can be referred to as a network of fibres.

As used herein, “integrated” or “integrally” refers to combining, coordinating or otherwise bringing together separate elements so as to provide a harmonious, consistent, interrelated whole. In the context of a textile, a textile can have various sections comprising networks of fibres with different structural properties. For example, a textile can have a section comprising a network of conductive fibres and a section comprising a network of non-conductive fibres. Two or more seconds comprising networks of fibres are said to be “integrated” together into a textile (or “integrally formed”) when at least one fibre of one network is interlaced with at least one fibre of the other network such that the two networks form a layer of the textile. Further, when integrated, two sections of a textile can also be described as being substantially inseparable from the textile. Here, “substantially inseparable” refers to the notion that separation of the sections of the textile from each other results in disassembly or destruction of the textile itself.

In some examples, conductive fabric (e.g., group of conductive fibres can be knit along with (e.g., to be integral with) the base fabric (e.g., surface) in a layer. Such knitting may be performed using a circular knit machine or a flatbed knit machine, or the like, from a vendor such as SANTONI® or STOLL™.

As used herein, “connected” or “coupled to” may include both direct coupling (in which two elements that are coupled to each other contact each other) and indirect coupling (in which at least one additional element is located between the two elements).

Although the disclosure has been described and illustrated in exemplary forms with a certain degree of particularity, it is noted that the description and illustrations have been made by way of example only. Numerous changes in the details of construction and combination and arrangement of parts and steps may be made. Except to the extent explicitly stated or inherent within the processes described, including any optional steps or components thereof, no required order, sequence, or combination is intended or implied. As will be will be understood by those skilled in the relevant arts, with respect to both processes and any systems, devices, etc., described herein, a wide range of variations and modifications are possible, and even advantageous, in various circumstances. The invention is intended to encompass all such variations and modification within its scope, as defined by the claims.

1. A method of sensing electrophysiological signals using dynamically changing lead configurations, the method comprising:
providing a garment system having a plurality of electrodes, each disposed to contact a respective body portion of a plurality of body portions of a wearer of the garment system;
sensing electrophysiological signals of the wearer in a first lead configuration of the garment system using a first subset of the electrodes;
detecting at least one change in contact location between the electrodes and the body portions; and
upon said detecting, sensing further electrophysiological signals of the wearer in a second lead configuration of the garment system using a second subset of the electrodes, wherein the second lead configuration provides an improved signal quality relative to the first lead configuration.

2. The method of claim 1, wherein the first subset of the electrodes and the second subset of the electrodes are the same.

3. The method of claim 1, wherein the first subset of the electrodes and the second subset of the electrodes are different.

4. The method of claim 1, wherein the change in contact location includes a change in contact of a given one of the electrodes from a given one of the body portions to a different one of the body portions.

5. The method of claim 1, wherein the change in contact location includes a hand of the body portions coming into contact with a particular one of the electrodes.

6. The method of claim 1, wherein said sensing electrophysiological signals includes sensing at least one of ECG, EEG, EMG, EOG, or bioimpedance signals.

7. The method of claim 1, further comprising:
establishing baseline readings for the wearer based on said sensing in the first lead configuration.

8. The method of claim 7, further comprising:
detecting a deviation from the baseline readings based on said sensing in the first lead configuration.

9. The method of claim 8, further comprising:
upon said detecting, providing a notification to the wearer to cause the garment system to transition to the second lead configuration.

10. A garment system for sensing electrophysiological signals using dynamically changing lead configurations, the system comprising:
a plurality of electrodes, each disposed to contact a respective body portion of a plurality of body portions of a wearer of the garment system;
wherein the garment system is operable to:
sense electrophysiological signals of the wearer in a first lead configuration using a first subset of the electrodes;
detect at least one change in contact location between the electrodes and the body portions; and
sense further electrophysiological signals of the wearer in a second lead configuration using a second subset of the electrodes, wherein the second lead configuration provides an improved signal quality relative to the first lead configuration.

11. The system of claim 10, wherein the first subset of the electrodes and the second subset of the electrodes are the same.

12. The system of claim 10, wherein the first subset of the electrodes and the second subset of the electrodes are different.

13. The system of claim 10, wherein at least one of the electrodes is disposed in a garment pocket.

14. The system of claim 10, wherein at least one of the electrodes is disposed on an interior garment surface.

15. The system of claim 10, wherein at least one of the electrodes is disposed on an exterior garment surface.

16. The system of claim 10, wherein at least one of the electrodes is disposed on a flap of a garment, wherein the flap is configured to be manipulated between a first position at an interior of the garment, and a second position at an exterior of the garment.

17. The system of claim 10, further comprising a contact surface for a hand of the wearer that is removably attachable to a garment.

18. The system of claim 10, further comprising a controller operable to process the electrophysiological signals and the further electrophysiological signals.

19. The system of claim 18, wherein the controller is communicatively coupled to at least one of the electrodes by way of an electrically conductive pathway.

20. The system of claim 18, wherein the electrically conductive pathway is formed of conductive yarn.

21. The garment system of claim 18, wherein the plurality of electrodes includes a textile electrode formed of conductive yarn.
